(19) United States
(12) Patent Application Publication
(10) Pub. No.: US 2024/0325281 A1
(43) Pub. Date: Oct. 3, 2024

(54) REACTIVE SKIN PROTECTANT CLEANING COMPOSITION FOR PROTECTING AGAINST CHEMICAL AGENT AND CLEANING METHOD USING THE SAME

(71) Applicant: AGENCY FOR DEFENSE DEVELOPMENT, Daejeon (KR)

(72) Inventors: Hae Eun JOE, Daejeon (KR); Chang Hwan KIM, Daejeon (KR); Dong Sung SEO, Daejeon (KR); Sang Woong KIM, Daejeon (KR); Young CHO, Daejeon (KR)

(21) Appl. No.: 18/613,856

(22) Filed: Mar. 22, 2024

(30) Foreign Application Priority Data
Mar. 29, 2023 (KR) 10-2023-0041197

(51) Int. Cl. A61K 8/86; A61K 8/03; A61K 8/27; A61K 8/28; A61K 8/34; A61K 8/9789; A61Q 19/10

(52) U.S. Cl. CPC A61K 8/86; A61K 8/03; A61K 8/27; A61K 8/28; A61K 8/345; A61K 8/9789; A61Q 19/10

(57) ABSTRACT

Provided are a multilayer cleaning agent composition including a perfluorinated oil, a hydrophilic functional group-bonded fluorine-based additive, and a polyol, and a method of cleaning a fluorine-based reactive skin protectant using the same. The composition may rapidly and safely clean a reactive skin protectant including the fluorine-based component which is applied to skin for preventing a chemical agent from penetrating into the skin and decomposition products thereof, thereby cleanly removing residues.

### CROSS-REFERENCE TO RELATED APPLICATIONS

This application claims priority under 35 U. S. C. § 119 to Korean Patent Application No. 10-2023-0041197, filed on Mar. 29, 2023, in the Korean Intellectual Property Office, the disclosure of which is incorporated herein by reference in its entirety.

### TECHNICAL FIELD

The following disclosure relates to a multilayer cleaning agent which rapidly and effectively cleans and removes the reactive topical skin protectant applied to the skin in order to prevent chemical agents from penetrating into the skin, and a cleaning method using the same.

### BACKGROUND

Chemical agents are known as a weapons of mass destruction (WMD) which may cause fatal damage to humans in very small amounts and even take away life. Since the chemical agents have been used as a means of killing in war since the German army used chlorine gas in World War I and may be used as a tool of terrorism as in the Tokyo subway Sarin incident in 1995, development of a defense against the chemical agents is desperately needed.

When exposed to the chemical agent, it is absorbed into the body through the respiratory tract or skin and causes fatal symptoms such as miosis, dyspnea, muscle cramp, genetic mutation, cancer, inflammation, and blistering, thereby destroying body tissue and even leading to death.

The Chemical agents include gaseous chocking agents and blood agents which are absorbed into the body through the respiratory tract, and nerve agents, such as Tabun, Sarin, Cyclo-Sarin, Soman, Novichok, which penetrate into the body through the skin or breathing in a liquid form, thereby cause death by blocking enzymes which decompose neurotransmitters, and blister agents, such as sulfur mustard, nitrogen mustard, Lewisite, which produce blisters on skin.

For gaseous chemical agents, it is the most effective to wear a gas mask with an air purifier to protect the respiratory tract. For liquid chemical agents, a defense method of covering face and exposed skin with protective clothing, film, and the like is mainly used, but has an insufficient defensive effect and limitations in use. Recently, studies on the topical skin protection composition as a cream or ointment form which is directly applied on the skin for protecting against liquid chemical agents are on the rise.

As a specific example, Korean Patent Registration No. 10-2455140 discloses a reactive topical skin protection composition including fluorine-based components, a detoxification active material, and the like. Further, Korean Patent Registration No. 10-0853443 discloses a reactive topical skin protection cream composition including glycerin, a detoxification active material, and the like.

Since these topical skin protection compositions of the prior arts do not have problems such as behavioral discomfort, dyspnea, and heat buildup which are shown when a gas mask or a protective clothing is worn for protecting against a chemical agents, they have less physical burden on the user, but since they are formulations in a form directly applied to the skin, there is an inconvenience to remove the skin protection composition after a certain period of time after application regardless of whether contaminated with chemical agents.

A glycerin-based skin protection composition disclosed in Korean Patent Registration No. 10-0853443 may be easily removed when washed with water, but a fluorine-based skin protection composition disclosed in Korean Patent Registration No. 10-2455140 has a unique characteristic of being not removed at all with water due to its water repelling effect, also not completely removed even with a cleaning component such as soap or surfactants.

After a certain period of time after applying the skin protection composition including the fluorine-based component having the unique characteristic to the skin, any cleaning agent which easily removes the fluorine-based skin protection composition present on the skin or the method thereof has not been developed until now.

### RELATED ART DOCUMENTS

### Patent Document

(Patent Document 1) Korean Patent Registration No. 10-2455140

(Patent Document 2) Korean Patent Registration No. 10-0853443

### SUMMARY

An embodiment of the present invention is directed to providing a multilayer cleaning agent which rapidly and effectively cleans and removes a reactive topical skin protectant, in particular, a fluorine-based skin protection composition, which is applied for preventing a chemical agent from penetrating into the skin.

In one general aspect, a multilayer cleaning agent composition includes: a perfluorinated oil, a hydrophilic functional group-bonded fluorine-based additive, and a polyol.

In an exemplary embodiment of the present invention, the perfluorinated oil may be one or more selected from the group consisting of polyperfluoroisopropyl ether, polyperfluoromethylisopropyl ether, perfluoroperhydrobenzyl tetralin, and polyperfluoroethoxymethoxy difluorohydroxyethyl ether.

In an exemplary embodiment of the present invention, the perfluorinated oil may be included at 20 to 80 wt % based on the total weight of the composition.

In an exemplary embodiment of the present invention, the hydrophilic functional group-bonded fluorine-based additive may be one or more selected from the group consisting of perfluoroethoxymethoxy difluoroethyl PEG phosphate, polyperfluoroethoxymethoxy PEG-2 phosphate, polyperfluoroethoxymethoxy difluoroethyl PEG ether, ammonium C9-10 perfluoroalkyl sulfonate, ammonium C6-16 perfluoroalkylethyl phosphate, TEA-C8-18 perfluoroalkylethyl phosphate, C14-18 perfluoroalkylethyl thiohydroxypropyltrimonium chloride, C8-18 fluoroalcohol phosphate, C9-13 fluoroalcohol, and C9-15 fluoroalcohol phosphate.

In an exemplary embodiment of the present invention, the fluorine-based additive may be included at 1 to 20 wt % based on the total weight of the composition.

In an exemplary embodiment of the present invention, the polyol may be one or more selected from the group consisting of butylene glycol, propylene glycol, glycerin, diglycerin, 1,2-pentanediol, 1,2-hexanediol, and 1,2-octanediol.

In an exemplary embodiment of the present invention, the polyol may be included at 1 to 20 wt % based on the total weight of the composition.

In an exemplary embodiment of the present invention, 0.1 to 2 wt % of water may be further included based on the total weight of the composition.

In an exemplary embodiment of the present invention, one or more plant extracts selected from the group consisting of a Portulaca oleracea extract, a Rhodiola rosea extract, a mugwort extract, a Zamioculcas zamiifolia extract, a black bean extract, a mung bean extract, an Asparagus cochinchinensis extract, a horsetail extract, a persimmon leaf extract, a potato extract, a poncirus extract, a calendula extract, an Oenothera odorata extract, a white atractylis extract, a hedge parsley extract, and an Houttuynia cordata extract may be further included.

In an exemplary embodiment of the present invention, the plant extract may be included at 0.1 to 5 wt % based on the total weight of the composition.

In an exemplary embodiment of the present invention, the composition may further include one or more inorganic particle powders selected from the group consisting of barium sulfate, magnesium oxide, magnesium hydroxide, magnesium carbonate, aluminum oxide, aluminum hydroxide, silica, calcium hydroxide, carbonate, titanium dioxide, titanium hydroxide, iron oxide, iron hydroxide, zinc oxide, zinc hydroxide, zirconium oxide, zirconium hydroxide, cerium oxide, and cerium hydroxide.

In an exemplary embodiment of the present invention, the inorganic particle powder may be included at 0.01 to 3 wt % based on the total weight of the composition.

In an exemplary embodiment of the present invention, the composition may be a multilayer type including 2 to 4 boundaries.

In another general aspect, a method of cleaning a fluorine-based reactive skin protectant includes bringing the multilayer cleaning agent composition described above into contact with the skin to which a fluorine-based reactive skin protectant has been applied.

Other features and aspects will be apparent from the following detailed description, the drawings, and the claims.

### BRIEF DESCRIPTION OF THE DRAWINGS

FIG. 1 is an image showing a cleaning agent including perfluorinated oil/fluorine-based additive/polyol component, prepared according to Example 1 of the present invention. The cleaning agent included a first layer of a perfluorinated oil, a second layer of a hydrophilic functional group-bonded fluorine-based additive, and a third layer of a polyol layer including water.

FIG. 2 is an image showing a cleaning agent prepared according to Example 3 of the present invention. The cleaning agent included a first layer of a perfluorinated oil, a second layer of a fluorine-based additive, and a third layer of a layer in which zirconium was dispersed in a polyol.

FIG. 3 is photographs of four specimens of artificial leather on which the reactive topical skin protection composition was applied according to Experimental Example 1 of the present invention, which were manufactured for measuring a cleaning effect of the multilayer cleaning agent of the present invention.

FIG. 4 is images of four specimens manufactured according to Experimental Example 1-2 of the present invention, which were observed by an electron microscope.

FIG. 5 shows element mapping results for a cleaned specimen (C) of Example 4 performed according to Experimental Example 1-4 of the present invention.

FIG. 6 shows element mapping results for a specimen (D) of Comparative Example 2 from which the topical skin protection composition was removed by wiping with a cotton pad, performed according to Experimental Example 1-4 of the present invention.

### DETAILED DESCRIPTION OF EMBODIMENTS

Hereinafter, a multilayer cleaning agent composition according to the present invention and a method of cleaning a fluorine-based reactive skin protectant using the composition will be described in detail with reference to the attached drawings.

Technical terms and scientific terms used herein have the general meaning understood by a person skilled in the art unless otherwise defined, and a description for the known function and configuration which may unnecessarily obscure the gist of the present invention will be omitted in the following description and the accompanying drawings.

In addition, the terms used in the detailed description of the present invention are only for effectively describing a specific example, and are not intended to limit the present invention.

In addition, in describing constituent elements of the present invention, terms such as first, second, A, B, (a), and (b) may be used. These terms are used only to differentiate the constituent elements from other constituent elements, and the nature, sequence, order, or the like of the corresponding constituent elements is not limited by these terms.

In addition, units used in the present specification without particular mention are based on weights, and as an example, a unit of % or ratio may refer to a wt % or a weight ratio, and wt % may refer to wt % of any one component in a total composition, unless otherwise defined.

In addition, the numerical range used in the present specification may include all values within the range including the lower limit and the upper limit, increments logically derived in a form and span in a defined range, all double limited values, and all possible combinations of the upper limit and the lower limit in the numerical range defined in different forms. Unless otherwise particularly defined in the specification of the present invention, values which may be outside a numerical range due to experimental error or rounding off of a value may be also included in the defined numerical range.

In addition, the term “comprise” in the present specification is an open-ended description having a meaning equivalent to the term such as “is/are provided”, “contain”, “have”, or “is/are characterized”, and does not exclude elements, materials, or processes which are not further listed.

The name of each compound or component exemplified for describing the present invention follows the rules of International Union of Pure and Applied Chemistry (IUPAC) Nomenclature or International Nomenclature Cosmetic Ingredients (INCI).

Hereinafter, a multilayer cleaning agent composition which was contrived after performing an intensive study for efficiently cleaning and removing a reactive fluorine-based skin protectant which has been applied for preventing a chemical agent from penetrating into the skin will be described in detail.

A topical skin protection composition in a form of being applied to the skin so as to prevent a chemical agent which may be used as a means of war or terrorism from penetrating into the skin to cause critical damage has been developed. The reactive topical skin protection composition is classified into two types depending on the polarity of the compound, and a hydrophilic/polar skin protection composition may be cleaned often using water.

Hydrophobic contaminants such as oil strains are not cleanly removed when cleaned only with water, and may be cleanly removed only with a cleaning agent including a surfactant or an organic solvent component.

However, a strongly hydrophobic and non-polar fluorine-based topical skin protection composition may not be cleaned with water, and also, is difficult to be cleaned even with a cleaning agent composition including a hydrocarbon-based surfactant or an organic solvent component. Therefore, the multilayer cleaning agent composition of the present invention is formed so that a reactive topical skin protection composition including a supersaturated fluorine-based oil having strong hydrophobicity and non-polarity may be cleaned.

Specifically, the cleaning agent composition according to the present invention may include a perfluorinated oil, a hydrophilic functional group-bonded fluorine-based additive, and a polyol.

Herein, the perfluorinated oil may be one or more selected from the group consisting of polyperfluoroisopropyl ether, polyperfluoromethylisopropyl ether, perfluoroperhydrobenzyl tetralin, and polyperfluoroethoxymethoxy difluorohydroxyethyl ether. Preferably, it may be perfluoropolyether, but is not limited thereto. The perfluorinated oil may be included at 20 to 80 wt % or 30 to 75 wt % based on the total weight of the composition. In this case, cleaning may be performed by efficiently removing a fluorine-based compound which is a constituent component of a reactive skin protectant.

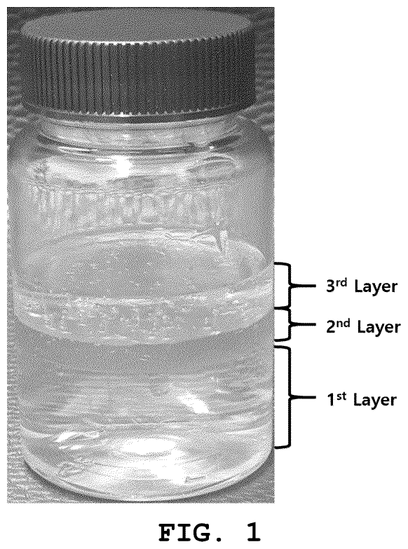

The perfluorinated oil is a complete non-polar liquid having a density of about 1.9 cm³/g, and is a component which is the heaviest of the constituent components used in the present invention and has lowest surface tension. Therefore, the perfluorinated oils are present in a first layer, as shown in FIG. 1.

The hydrophilic functional group-bonded fluorine-based additive may be included at 1 to 20 wt % or 3 to 18 wt % based on the total weight of the composition. By including the additive at the above content, emulsification or dispersion of a hydrophilic material including water may be economically accelerated, and thus, the hydrophilic material including water may contribute to hydrolysis reaction acceleration of a chemical agent.

Specifically, it is appropriate for the fluorine-based additive to maintain an emulsified or dispersed state only for a short time required for cleaning the fluorine-based reactive topical skin protection composition, for example, for several minutes to several hours during cleaning, and when the fluorine-based additive is included at the above content, it is rather preferred to maintain the emulsified or dispersed state for a short time.

In the present invention, the hydrophilic functional group may be one or more selected from the group consisting of a phosphate group, a carboxyl group, a sulfonate group, an ester group, a polyethylene glycol group, an amide group, an amine group, and a hydroxyl group.

Specifically, the hydrophilic functional group-bonded fluorine-based additive may be one or more selected from the group consisting of perfluoroethoxymethoxy difluoroethyl PEG phosphate, polyperfluoroethoxymethoxy PEG-2 phosphate, polyperfluoroethoxymethoxy difluoroethyl PEG ether, ammonium C9-10 perfluoroalkyl sulfonate, ammonium C6-16 perfluoroalkylethyl phosphate, TEA-C8-18 perfluoroalkylethyl phosphate, C14-18 perfluoroalkylethyl thiohydroxypropyltrimonium chloride, C8-18 fluoroalcohol phosphate, C9-13 fluoroalcohol, and C9-15 fluoroalcohol phosphate.

The cleaning agent composition according to the present invention is a hydrophilic material, and includes the polyol having two or more alcohol functional groups such as diol or triol. Since a primary alcohol such as ethanol rather promotes skin penetration of the chemical agent during a process of cleaning the fluorine-based reactive skin protectant, it is not preferred. Specifically, the polyol may be one or more selected from the group consisting of butylene glycol, propylene glycol, glycerin, diglycerin, 1,2-pentanediol, 1,2-hexanediol, and 1,2-octanediol. The polyol may be used with water, and the two may be mixed with each other as polar molecules and form one layer. Herein, water may be included at 0.1 to 2 wt % or 0.5 to 1.5 wt % based on the total weight of the composition.

The cleaning agent composition of the present invention may exert excellent cleaning ability for the fluorine-based reactive skin protectant according to a combination of the perfluorinated oil, the hydrophilic functional group-bonded fluorine-based additive, and the polyol described above. Specifically, according to the present invention, a hydrophilic material such as water or polyol is easily released from the inside of micelles in a cleaning process rather than is stably confined in emulsified micelles by the fluorine-based additive, thereby hydrolyzing a chemical agent which may move to a coating film of the reactive skin protectant and be contaminated.

In a preferred exemplary embodiment, the cleaning agent composition of the present invention may exist as a three-layer structure which is divided into a perfluorinated oil layer, a hydrophilic functional group-bonded fluorine-based additive layer, and a polyol layer. Since the fluorine-based additive has lower density than the fluorine-based oil, higher density than water and a hydrophilic material, and lower surface tension, it may be present in a middle layer without being mixed with each material separated in upper and lower layers unless the composition is vigorously shaken or kinetic energy is applied from the outside. This may be confirmed as a second layer shown in FIG. 1.

As an example, physical properties of each specific component are shown in the following Table 1:

TABLE 1

| Physical | | | Perfluorinated |
|---|---|---|---|
| properties\Component | Water | Glycerin | polyether |
| Polarity | Polar | Polar | Non-polar |
| Density (g/cm³) | 1.0 | 1.26 | ~1.9 |
| Surface tension | 72 | 63 | ~20 |
| (dyn/cm) | | | |

Water and glycerin are polar molecules, and glycerin has higher density, but water has higher surface tension, and these two may be mixed with each other to form one layer. However, since the perfluorinated polyether is a non-polar molecule and is not mixed with water or glycerin, when these are combined, they are separated to form a layered structure. Herein, since the perfluorinated polyether is one of the compounds having the highest density except mercury which is liquid at room temperature and has much higher density than water and glycerin, a layer including the perfluorinated polyether is positioned under the layer including water and glycerin.

The hydrophilic functional group-bonded fluorine-based additives may show characteristics of being separated without being mixed due to differences in density and surface tension from the perfluorinated oil layer and maintaining the layer. Specifically, the fluorine-based additive has a density of 1.7 cm³/g or less and the density may be shown lower than that of the perfluorinated oil layer.

The composition which is combined according to the physical properties, polarity, and interfacial chemical properties of each constituent component of the present invention maintains the multilayer form, as shown in FIG. 1. That is, an exemplary embodiment of the present invention may be a three-layer cleaning agent composition including a first layer of the perfluorinated oil, a second layer of the hydrophilic functional group-bonded fluorine-based additive, and a third layer of the polyol including water.

Herein, the factor maintaining the multilayer type may be a difference in density of liquid components, and heavier components are positioned in a lower layer. The factor causing liquid components in contact with each other to be separated into clean interfaces and exist may be effects from the density, difference in polarity, and surface tension of each liquid component.

The multilayer cleaning agent composition according to the present invention uses the density, difference in polarity, and surface tension, whereby layer separation is performed at a rapid speed when being brought into contact with the fluorine-based reactive skin protectant and a hydrolysis reaction or cleaning action for detoxification is rapidly performed.

In addition, the present invention may use a plant extract for the purpose of soothing the skin after cleaning by relieving, neutralizing, and converging skin irritation which may be caused by mechanical friction applied during a process of cleaning a fluorine-based skin protectant or irritating substances, for example, a trace amount of chemical agent hydrolysis by-product by water or a hydrophilic material.

A specific example of the plant extract may be one or more selected from the group consisting of a Portulaca oleracea extract, a Rhodiola rosea extract, a mugwort extract, a Zamioculcas zamiifolia extract, a black bean extract, a mung bean extract, an Asparagus cochinchinensis extract, a horsetail extract, a persimmon leaf extract, a potato extract, a poncirus extract, a calendula extract, an Oenothera odorata extract, a white atractylis extract, a hedge parsley extract, and an Houttuynia cordata extract. The plant extract may be included at 0.1 to 5.0 wt % or 1 to 4 wt % based on the total weight of the composition.

The plant extract may be mixed with the polyol or water which are used as a hydrophilic material. Since the mixtures have lower density and higher surface tension than the perfluorinated oil and the fluorine-based additive, they may be present in a third layer which is the topmost layer, as shown in FIG. 1.

In addition, the present invention may use inorganic particle powder for removing the chemical agent which may contaminate the skin protectant with a very small amount by accelerating a decomposition reaction more easily than hydrolyzing the chemical agent with water or a hydrophilic material. These inorganic particle powders may be a catalyst of a decomposition reaction of the chemical agent, and may be effective for a decomposition reaction by adsorbing the chemical agent which may contaminate the skin protectant and be present in a small amount.

The inorganic particle powder may be, specifically, one or more selected from the group consisting of barium sulfate, magnesium oxide, magnesium hydroxide, magnesium carbonate, aluminum oxide, aluminum hydroxide, silica, calcium hydroxide, calcium carbonate, titanium dioxide, titanium hydroxide, iron oxide, iron hydroxide, zinc oxide, zinc hydroxide, zirconium oxide, zirconium hydroxide, cerium oxide, and cerium hydroxide. It is preferred that the inorganic particle powder is used in a range of 0.01 to 3 wt % or 0.1 to 2.5 wt %.

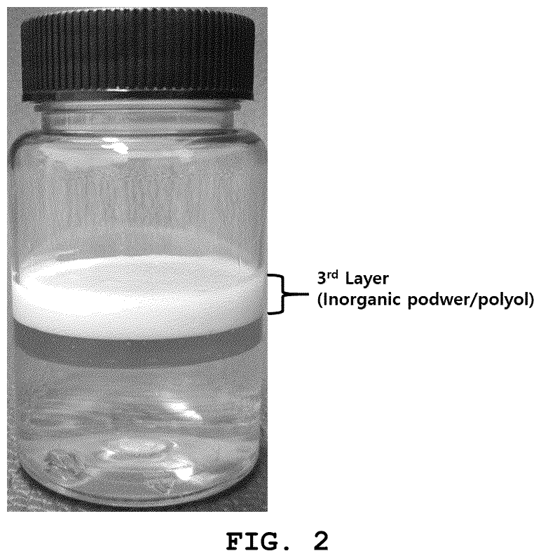

The particle size of the inorganic particle powder may vary depending on the type and shape, but an average particle diameter may be preferably 0.05 to 5.0 μm. Since the inorganic particle powder is hydrophilic, it is present in the third layer shown in FIG. 2. Herein, since the density of the inorganic particle powder is usually higher than the density of water or the polyol which is a third layer liquid, the inorganic particle powder slowly settles down over time, so that it gathers in the upper interface of the fluorine-based additive which is the second layer, so that the third layer may be divided into an inorganic particle powder sedimentation layer and a hydrophilic liquid layer. Therefore, it may be shown as a four-layer cleaning agent.

Since the four-layer cleaning agent is easily dispersed again when shaken for cleaning like the three-layer cleaning agent, the coating film of the fluorine-based skin protectant to be desired in the present invention may be effectively removed by cleaning.

Magnesium oxide is a detoxification active material and used as a component of a reactive skin protectant, and since the magnesium oxide adsorbs the chemical agent or may include decomposition by-products which is not beneficial to the human body depending on the detoxification reaction, it is preferred to necessarily remove magnesium oxide by rapid cleaning. Therefore, a technique of cleaning so that the magnesium oxide does not remain is needed, and the present invention exerts excellent cleaning ability so that there is no remaining magnesium oxide.

In order to clean the coating film of the reactive skin protectant applied for preventing the chemical agent, the present invention may be the following multilayer cleaning agent compositions:

First, it may be a two-layer cleaning agent in the case of a combination including fluorine-based oil/fluorine-based additive, fluorine-based oil/polyol, or fluorine-based oil/water or plant extract.

Second, it may be a three-layer cleaning agent in the case of a combination including fluorine-based oil/fluorine-based additive/polyol, water, or plant extract.

Third, it may be a four-layer cleaning agent in the case of a combination in which the inorganic particle powder is dispersed in the third layer of the third layer-type cleaning agent.

In another general aspect, a method of cleaning a fluorine-based reactive skin protectant includes bringing the multilayer cleaning agent composition described above into contact with the skin to which a fluorine-based reactive skin protectant has been applied.

Hereinafter, the present disclosure will be described in more detail by the following examples. However, the following examples are only a reference for describing the present disclosure in detail, and the present disclosure is not limited thereto and may be implemented in various forms.

### Examples 1 to 4

Raw materials according to the composition of each example of the following Table 2 were added in order to a 200 ml beaker, mixed well, and transferred to a transparent container to prepare a multilayer cleaning agent.

TABLE 2
Multilayer cleaning agent composition (unit: wt %)

| | Perfluorinated | | Fluorine-based | | | | | | | | | Plant | Inorganic | |
|---|---|---|---|---|---|---|---|---|---|---|---|---|---|---|
| | oil | | additive | | | | Polyol | | | Water | | extract | particle powder | |
| | A-1 | A-2 | B-1 | B-2 | B-3 | B-4 | C-1 | C-2 | C-3 | D | E-1 | E-2 | F-1 | F-2 |
| Example 1 | 70 | | 15 | | | | 14 | | | 1.0 | | | | |
| Example 2 | 50 | 20 | 10 | 5 | | | 10 | 3.0 | | | | | | |
| Example 3 | 10 | 50 | 5.0 | | | 10.0 | 10.0 | | | | 1.5 | 1.0 | 2.5 | |
| Example 4 | 5 | 70 | 5.0 | | 5.0 | | 5.0 | | 5.0 | | 0.5 | 2.0 | 1.5 | 1.0 |

A-1: polyperfluoromethylisopropyl ether
A-2: polyperfluoroisopropyl ether
B-1: polyperfluoroethoxymethoxy difluoroethyl PEG phosphate
B-2: ammonium C6-16 perfluoroalkylethyl phosphate
B-3: ammonium C9-10 perfluoroalkyl sulfonate
B-4: C8-18 fluoroalcohol phosphate
C-1: glycerin
C-2: 1,2-hexanediol
C-3: butylene glycol
D: water
E-1: Portulaca oleracea extract
E-2: mugwort extract
F-1: zirconium hydroxide
F-2: zinc oxide

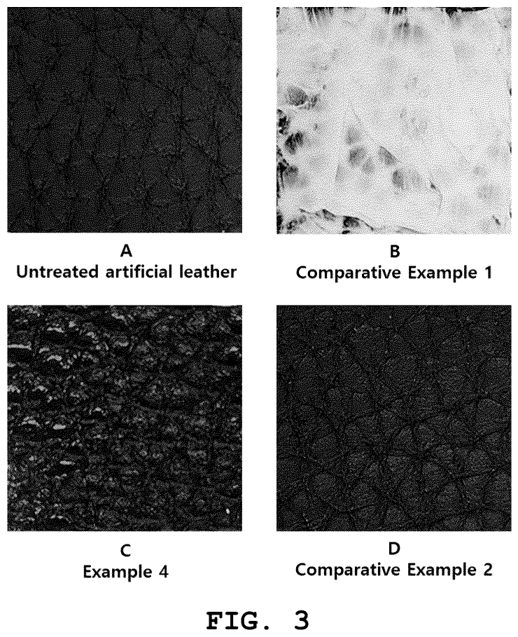

The cleaning agents prepared according to Examples 1 to 4 were all shown to be a three-layer type. Images of the cleaning agents according to Examples 1 and 3 are shown in FIGS. 1 and 3, respectively. In FIG. 1, a polyol layer formed the third layer, and in FIG. 3, a layer in which zirconium hydroxide was dispersed formed the third layer.

### Preparation Example 1

A fluorine-based reactive topical skin protection composition was prepared according to the composition shown in the following Table 3 and used as a test comparative example for evaluating the cleaning effect of the multilayer cleaning agent of the present invention.

TABLE 3
topical Skin protection composition including
fluorine-based component (unit: wt %)

| | | Comparative |
|---|---|---|
| No. | Raw material name (INCI) | Example 1 |
| 1 | Polyperfluoromethylisopropyl ether | 55.0 |
| 2 | Perfluoropolyethylene | 25.0 |
| 3 | Magnesium oxide surface-treated | 20.0 |
| | with perfluoroalkyl | |

### Experimental Example 1. Evaluation of Cleaning Effect of Multilayer Cleaning Agent Using Artificial Leather

Artificial leather for performing an evaluation test instead of human skin was selected, and specimens on which the reactive skin protectant including the fluorine-based component prepared according to Preparation Example 1 was applied were manufactured.

### 1-1. Manufacture of Specimen for Test Analysis

Four specimens obtained by cutting artificial leather of an average thickness of 0.95 mm having an irregular pattern shown in A of FIG. 3 into a size of 2 cm×2 cm horizontally and vertically were prepared. The artificial leather specimens had an average weight of 0.21 g. One (indicated as A in FIG. 3) of the 4 artificial leather specimens was not treated at all, and to the remaining three specimens, the reactive topical skin protection composition including the fluorine-based component of Preparation Example 1 quantified at 0.45 g each was evenly applied on an area of 2 cm×2 cm using a stainless-steel spatula.

### Comparative Example 1

One (indicated as B in FIG. 3) of the three specimens which the reactive skin protectant according to Preparation Example 1 was applied was left as it was without a cleaning treatment.

### Example 4

One (indicated as C in FIG. 3) of the remaining two specimens was cleaned with the three-layer cleaning agent of Example 1 of the present invention.

### Comparative Example 2

The remaining specimen (indicated as D in FIG. 3) was wiped with a cotton pad to remove a coating film.

### 1-2. Analysis of SEM Image of Each Specimen

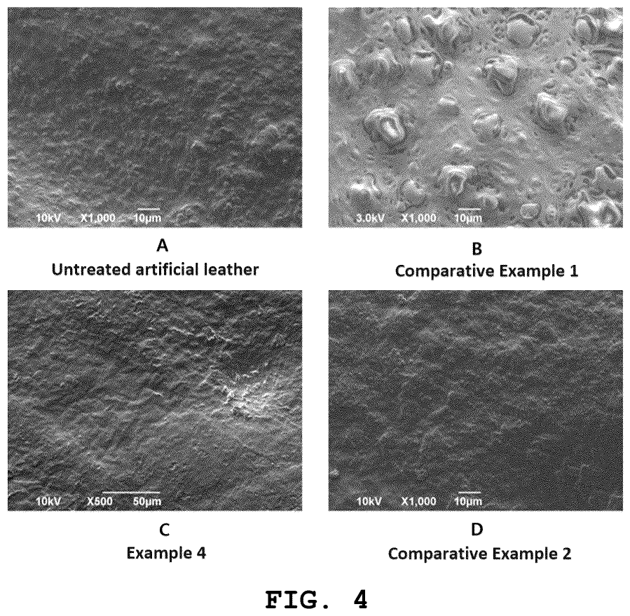

Photographs of four specimens A, B, C, and D, each of which had completed treatment, observed at 1,000 times magnification using an electron microscope (SEM) are shown in FIG. 4.

In the SEM images, it is shown that A had small irregularities like wrinkles or folds in the skin. In Comparative Example 1, the coating layer of the skin protectant prepared according to Preparation Example 1 in a thick form was shown and combined inorganic particles combined with the fluorine-based component were locally observed. In Example 4, it is shown that irregularities of the artificial leather were a little changed depending on the cleaning process. It is considered that the artificial leather was a little swollen. In Comparative Example 2, it is considered that the irregularities were changed due to friction by pressure on the irregularities of the artificial leather in the process of removing a coating layer by wiping with a cotton pad.

### 1-3. EDS Analysis of Each Specimen

The results of analyzing the elemental composition ratio of four specimens used in the SEM image analysis in the above 1-2 by EDS are shown in the following Table 4:

TABLE 4
Elemental composition ratio (wt %) of each specimen

| | A | B* | | D |
|---|---|---|---|---|
| Element\ | (Artificial | Comparative | C | Comparative |
| Specimen | leather) | Example 1 | Example 4 | Example 2 |
| Carbon | 100 | 20.15 | 86.51 | 78.72 |
| Fluorine | Not | 70.57 | 13.49 | 19.88 |
| | detected | | | |
| Magnesium | Not | 9.28 | Not | 1.41 |
| | detected | | detected | |

*Since Specimen B was coated with the fluorine-based component and was a complete non-conductor, it was analyzed after being coated with Au, while Specimens A, C, and D were analyzed without Au coating.

In the elemental analysis results in Table 4, it is confirmed that since the material used for manufacturing artificial leather was an organic compound, only a carbon element was detected in artificial leather Specimen A which was not treated at all. In Specimen B corresponding to Comparative Example 1 and Specimen D corresponding to Comparative Example 2, it is confirmed that carbon, fluorine, and magnesium atoms were all detected.

In Comparative Example 2, 1.41 wt % of magnesium was detected, which means that though the coating layer appeared to have been removed, magnesium oxide particles which were fine powder combined with the fluorine-based component remained on the surface of the artificial leather. That is, removal by wiping the coating film with a cotton pad, tissue, non-woven fabric, or the like may be very dangerous since it may cause secondary contamination to the skin.

Unlike this, in Specimen C corresponding to Example 4, carbon and fluorine atoms were detected, but a magnesium atom of magnesium oxide which was combined at 20.0 wt % with the fluorine-based component was not detected. The analysis results mean that magnesium oxide was completely removed without remaining in the artificial leather by the cleaning effect of the cleaning agent of the present invention. Thus, since the multilayer cleaning agent of the present invention completely cleaned and removed the coating film of the reactive skin protectant including the fluorine-based component, safety from secondary contamination from the chemical agent is confirmed.

### 1-4. Elemental Mapping of Each Specimen

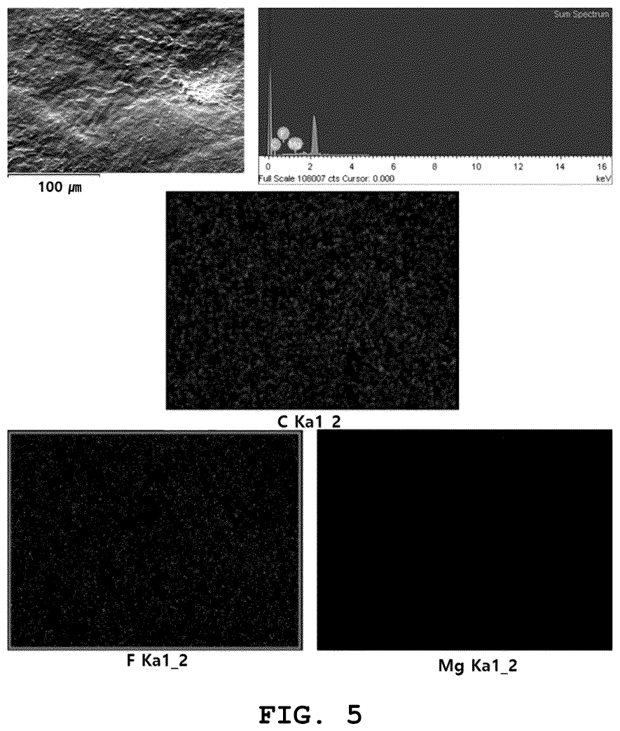

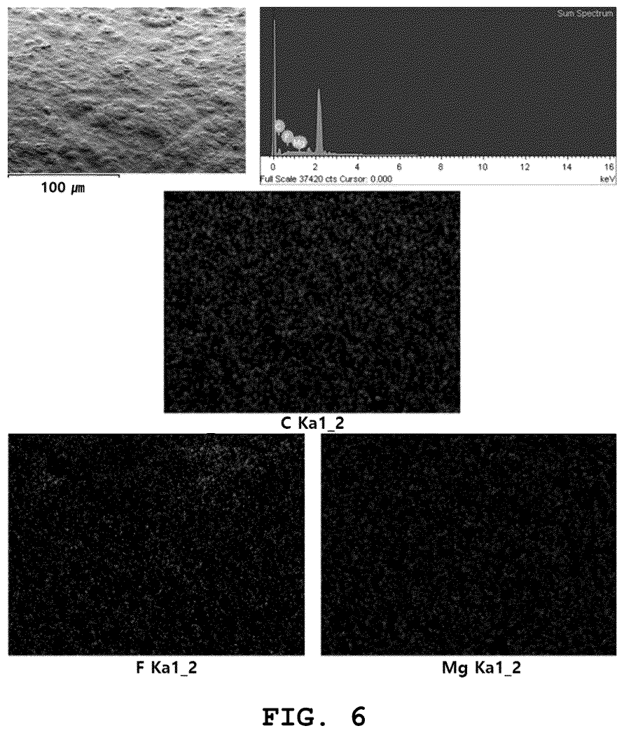

In order to confirm whether magnesium oxide which was fine inorganic powder included in the reactive topical skin protection composition of Preparation Example 1 was removed from the artificial leather specimen, elemental mapping on Specimens C and D was performed by EDS. The results are shown in FIGS. 5 and 6.

The mapping results of the magnesium element of Example 4 are shown in a bottom right drawing (indicated as Mg Ka1_2) of FIG. 5. As shown in FIG. 5, a magnesium element was not confirmed.

However, in the mapping results of the magnesium element of Comparative Example 2 shown in the bottom right drawing (indicated as Mg Ka1_2) of FIG. 6, magnesium residue is confirmed from scattered red dots. Therefore, it was found that when the reactive skin protectant is removed by wiping with a cotton pad, residue remains in an invisible state.

From the evaluation test results, it is confirmed that the multilayer cleaning agent of the present invention provides an excellent effect in cleaning the reactive skin protectant including the fluorine-based component and removing the residue.

The present invention may provide a multilayer cleaning agent which rapidly and safely cleans and removes a reactive skin protectant including a fluorine-based component which is applied to the skin for preventing a chemical agent from penetrating into the skin and decomposition products thereof.

What is claimed is:

1. A multilayer cleaning agent composition comprising: a perfluorinated oil, a hydrophilic functional group-bonded fluorine-based additive, and a polyol.

2. The multilayer cleaning agent composition of claim 1, wherein the perfluorinated oil is one or more selected from the group consisting of polyperfluoroisopropyl ether, polyperfluoromethylisopropyl ether, perfluoroperhydrobenzyl tetralin, and polyperfluoroethoxymethoxy difluorohydroxyethyl ether.

3. The multilayer cleaning agent composition of claim 2, wherein the perfluorinated oil is comprised in an amount of 20 to 80 wt % based on the total weight of the composition.

4. The multilayer cleaning agent composition of claim 1, wherein the hydrophilic functional group is one or more selected from the group consisting of a phosphate group, a carboxyl group, a sulfonate group, an ester group, a polyethylene glycol group, an amide group, an amine group, and a hydroxyl group.

5. The multilayer cleaning agent composition of claim 1, wherein the hydrophilic functional group-bonded fluorine-based additive is one or more selected from the group consisting of perfluoroethoxymethoxy difluoroethyl PEG phosphate, polyperfluoroethoxymethoxy PEG-2 phosphate, polyperfluoroethoxymethoxy difluoroethyl PEG ether, ammonium C9-10 perfluoroalkyl sulfonate, ammonium C6-16 perfluoroalkylethyl phosphate, TEA-C8-18 perfluoroalkylethyl phosphate, C14-18 perfluoroalkylethyl thiohydroxypropyltrimonium chloride, C8-18 fluoroalcohol phosphate, C9-13 fluoroalcohol, and C9-15 fluoroalcohol phosphate.

6. The multilayer cleaning agent composition of claim 5, wherein the fluorine-based additive is comprised in an amount of 1 to 20 wt % based on the total weight of the composition.

7. The multilayer cleaning agent composition of claim 1, wherein the polyol is one or more selected from the group consisting of butylene glycol, propylene glycol, glycerin, diglycerin, 1,2-pentanediol, 1,2-hexanediol, and 1,2-octanediol.

8. The multilayer cleaning agent composition of claim 7, wherein the polyol is comprised in an amount of 1 to 20 wt % based on the total weight of the composition.

9. The multilayer cleaning agent composition of claim 1, further comprising: 0.1 to 2 wt % of water based on the total weight of the composition.

10. The multilayer cleaning agent composition of claim 1, further comprising: one or more plant extracts selected from the group consisting of a Portulaca oleracea extract, a Rhodiola rosea extract, a mugwort extract, a Zamioculcas zamiifolia extract, a black bean extract, a mung bean extract, an Asparagus cochinchinensis extract, a horsetail extract, a persimmon leaf extract, a potato extract, a poncirus extract, a calendula extract, an Oenothera odorata extract, a white atractylis extract, a hedge parsley extract, and an Houttuynia cordata extract.

11. The multilayer cleaning agent composition of claim 10, wherein the plant extract is comprised in an amount of 0.1 to 5 wt % based on the total weight of the composition.

12. The multilayer cleaning agent composition of claim 1, further comprising: one or more inorganic particle powders selected from the group consisting of barium sulfate, magnesium oxide, magnesium hydroxide, magnesium carbonate, aluminum oxide, aluminum hydroxide, silica, calcium hydroxide, calcium carbonate, titanium dioxide, titanium hydroxide, iron oxide, iron hydroxide, zinc oxide, zinc hydroxide, zirconium oxide, zirconium hydroxide, cerium oxide, and cerium hydroxide.

13. The multilayer cleaning agent composition of claim 12, wherein the inorganic particle powder is comprised in an amount of 0.01 to 3 wt % based on the total weight of the composition.

14. The multilayer cleaning agent composition of claim 1, wherein the composition includes 2 to 4 boundaries.

15. A method of cleaning a fluorine-based reactive skin protectant, comprising bringing the multilayer cleaning agent composition of claim 1 into contact with skin on which a fluorine-based reactive skin protectant has applied.
